(19) United States
(12) Patent Application Publication
(10) Pub. No.: US 2017/0197894 A1
(43) Pub. Date: Jul. 13, 2017

(54) PROCESS FOR PRODUCTION OF METHANOL

(71) Applicant: Haldor Topsøes A/S, Kgs. Lyngby (DK)

(72) Inventor: Ib DYBKJÆR, Copenhagen (DK)

(73) Assignee: Haldor Topsøes A/S, Kgs. Lyngby (DK)

(21) Appl. No.: 15/304,891

(22) PCT Filed: Apr. 24, 2015
(86) PCT No.: PCT/EP2015/058936

§ 371 Date: Oct. 18, 2016

(30) Foreign Application Priority Data
Apr. 29, 2014 (DK) PA 2014 00236

(51) Int. Cl. C07C 29/151; C01B 3/12; B01D 53/26; B01J 19/00; B01D 53/22; B01D 53/047; C07C 29/152; C01B 3/56

(52) U.S. Cl. CPC C07C 29/1518; C07C 29/152; C01B 3/12; C01B 3/56; B01J 19/0013; B01D 53/22; B01D 53/047; B01D 53/265; C01B 2203/0283; C01B 2203/042; C01B 2203/061; C01B 2203/0872; C01B 2203/0805; B01J 2219/00051; B01D 2256/16; B01D 2257/502; B01D 2257/504; B01D 2257/11; B01D 2257/7025; B01D 2257/40

(57) ABSTRACT

The present application relates to a process comprising the steps of—providing a purge stream from a synthesis section,—preheating at least part of said purge stream,—adding steam to the preheated purge stream to obtain a first mixed stream,—passing the first mixed stream through a shift conversion step thereby obtaining a conversion product stream,—passing at least part of the conversion product stream through a H₂ separation step producing a H₂ enriched stream and a H₂ depleted waste stream, and —returning at least part of said H₂ enriched stream to and/or upstream the synthesis section.

### BACKGROUND

In known methanol production plants and processes carbon oxides (CO and CO₂) are removed from the synthesis section together with other gaseous components in a purge stream in order to obtain/maintain preferred reaction conditions in the plant.

In known plants and processes this step may be relatively expensive due to the means needed in order to separate H₂ from the purge for recycling to the synthesis section.

Producing methanol theoretically requires a synthesis gas (syngas) with a module M equal to 2. The module M defined as

M=(H₂−CO₂)/(CO+CO₂)

As syngas typically also contains inert compounds, the optimum module may become slightly higher than 2, typically 2.05, allowing purge of the inerts which inevitable also will result in purge of reactants, H₂, CO and CO₂.

For a syngas with a module less than the optimum module as defined above, surplus carbon oxides are present, and the module must be adjusted to the required level, e.g. by recovery of H₂ from the purge stream and recycle of the recovered H₂ the synthesis section. In known processes this is done by recovering H₂ from the purge in a separation unit, e.g. a PSA unit or a membrane unit, which produces a H₂-enriched gas for recycle and a H₂-depleted waste gas.

Typically, part of the H₂ and the main part of the carbon oxides are lost with the waste gas.

In some known processes, the purge requirements are dictated by the requirement to control the content of inert compounds (mainly CH₄, N₂ and Ar) in the reaction gas in the synthesis section. In other known processes, the purge requirements are dictated by the need to provide sufficient H₂ for adjustment of the module in the synthesis section. In the latter case, it is desirous to increase the content of H₂ available for recycle by increasing the amount of H₂ obtained by the separation process, thus reducing the required purge and thereby increasing the production of methanol in the synthesis section.

### ASPECTS

In a first aspect of the present invention is provided a plant and process which reduce the purge, thereby increasing the production of e.g. methanol and reducing the requirements for the H₂ separation means.

In a second aspect of the present invention is provided a plant and process which enable the use of a less costly H₂ separation means using less energy.

In a third aspect of the present invention is provided a plant and process which increase the efficiency of converting a syngas to methanol as less reactants are lost.

In a fourth aspect of the present invention is provided a plant and process which allows use of a syngas feed with a module M lower than 2.

### SUMMARY OF THE INVENTION

These and other advantages are achieved by a process for recovering H₂ from the purge gas for recycle to the synthesis section with minimum loss of reactants from a production section, said process comprising the steps of
providing a purge stream from a synthesis section,
preheating at least part of said purge stream, adding steam, and passing the mixture through a shift conversion unit thereby obtaining a purge product stream
cooling the purge product stream and separating condensed water, to create a cooled, H₂ enriched first product stream,
passing at least part of the H₂ enriched first product stream to a H₂ separation step,
in the H₂ separation step producing a H₂ rich stream and a H₂ depleted waste stream, and
returning at least part of said H₂ rich stream to and/or upstream the synthesis section.

The H₂ depleted gas can be let out of the separation step via a waste gas line and may for example be used as fuel in fired heaters in the process plant or e.g. for power production.

Thus the present invention provides a process which ensures an increased yield of H₂ in the separation step/unit, thereby decreasing the required purge. In other words, the present process increases the production of methanol by reducing the loss of reactants. The applicant has shown that even though the introduction of a shift conversion section traditionally may be considered an expensive and thus undesirable step unlikely to be used in relation to e.g. purge gases, the result in form of an increased production in the present setup is significant and thus the overall result is desirable.

In preferred embodiments the purge stream is obtained from one or more processes including a methanol process, where the one or more processes are carried out based on a process stream such as syngas.

The purge stream may comprise CO, CO₂, H₂ and inerts. Said inerts can comprise but are not limited to CH₄, Ar, N₂, He.

If the synthesis section produces methanol the reactions preferably are:

CO+2H₂->CH₃OH and/or

CO₂+2H₂->CH₃OH+H₂O

In some embodiments the purge stream is passed to the shift conversion step essentially without pressure drop, i.e. at a pressure P₁ between 30-200 atm, between 50-110 e.g. between 30-60, between 60-100, between 100-200 atm.

The stream is preheated preferably to a temperature between 100-400 C., 150-300, between 175-250 C., between 180-220C., steam can be added in an amount corresponding to a steam to dry gas ratio of 0.1-5, 0,3-2, 0,5-1.

In other embodiments, the pressure of the purge gas is reduced before the shift conversion to a pressure less than 100 atm, less than 50 atm, between 15 and 40 atm. The resulting stream is preheated and steam is added in an amount corresponding to a steam to dry gas ratio of 0.1-5, 0,3-2, 0,5-0.1. The mixed gas is passed to the shift conversion step at a temperature 100-400 C., 150-300, between 175 - 250 C such as between 180-220 C.

After the shift conversion step, the gas may be cooled to below its dew point where after condensate is separated, and the gaseous stream passed to a H₂ separation unit such as a membrane unit or a PSA unit.

The H₂ separation unit such as a membrane unit or a PSA unit produces a H₂-enriched stream, which is returned to the synthesis process, and a H₂-depleted waste gas.

The temperature in the shift conversion step is preferably adjusted to the preheat temperature before the addition of steam.

When H₂ is returned to the feed mixing point the module M for the process, such a methanol production process, may be controlled thereby allowing an optimization of one or more of the processes in the production step/section.

Preferably the process stream is mixed with H₂ to achieve a module around M=2. In case of a module larger than 2 the reaction section contains excess H₂ and in case of a module lower than 2 the reaction section contains excess CO₂. Known methanol processes requires a syngas feed with a module between 1.0 and 100, typically between 1.9 and 3.0.

The present invention may allow processing of feed, such as a synthesis gas, with a module of less than that of the module desired for the production such as a module less than 2 for a methanol production. Some feeds and reforming processes may provide a syngas with a low module M. As an example auto thermal reforming ATR, may result in such a low module syngas. As the present process enhance the amount of H₂ recovered from the purge compared to traditional setups it may advantageously be applied in relation to such low module syngas. Also due to the enhanced H₂ recovery less purge needs to be taken out which may result in an increase in production in the synthesis section. Especially in the case of methanol production the decreased purge and increased H₂ recovery may be advantageous.

Thus the present process allows efficient methanol production from low module syngas such as syngas provided by ATR.

The invention further relates to a plant comprising
a synthesis section comprising a product separation unit providing a purge stream comprising H₂, a purge gas treatment line arranged to bring a mixture of purge gas and steam in contact with a shift conversion catalyst at proper conditions, and further comprising a cooler and a condensate separation unit, and
a gas separation unit for obtaining of a H₂ enriched gas for recycle to the synthesis section.

Thus the present invention provides a plant which by simple means allows for reduction of the loss of reactants carbon oxides and H₂.

The condensate separation unit is arranged downstream the synthesis section and upstream the gas separation unit.

The condensate separation unit provides a condensate stream and a vapor stream, which vapor stream comprises H₂.

The gas separation unit provides a H₂ enriched stream and a H₂ depleted stream. The H₂ enriched stream is brought back to the synthesis section via a H₂ line which H₂ line in some setups may comprise a compressor. The at least part of the H₂ enriched stream is added in the synthesis section at one or more mixing points.

The gas separation unit may for example comprise a membrane or be a PSA.

Preferably the plant is of the kind comprising units arranged to process a syngas stream in form of CO, CO₂, H₂ and inerts comprising but not limited to CH₄, Ar, N₂, He. Preferably the plant is arranged to process a syngas stream with a module M between 1.0 and 2.1. By the present method and plant it is possible to process a feed with a lower module than if known processes are used.

The synthesis section may comprise different units e.g. a syngas feed unit mixing syngas feed and recycled H₂ containing gas, heating means, one or more reaction units converting syngas to Methanol as well as a cooling unit wherein the reaction product is condensed to liquid Methanol.

The plant comprises a H₂ line arranged to bring H₂ from the purge gas treatment section to one or more syngas feed mixing points in the synthesis section and/or upstream the synthesis section (e.g. a methanol production section). The H₂ recovered from the purge gas by the present method can be used to adjust the module M of the process gas to a desired value.

The present process and plant thus provides a treated purge gas with increased H₂ content compared to the untreated purge and reduced CO content (i.e. the H₂ enriched stream). This means that the requirements to the means for isolating at least H₂ from gas outlet/the purge section may be less strict when compared to known plants while still obtaining an increased H₂ concentration in the H₂ line.

The process as described herein is preferably carried out on a plant as also described. Arguments and described advantages of common features and corresponding steps/units in the process/plant respectively may apply to both step/unit where appropriate even if only described in relation to one of step/unit or plant part.

The present method and plant may be particularly useful where the feed provided to the synthesis section is a synthesis gas made by auto thermic reforming, ATR at a low Steam/Carbon ratio.

Thus by the present invention is provided a method and a plant to increase the yield of H₂ recovered from the purge stream, thus reducing the required purge. The reduced purge results in an increased production of methanol in the synthesis section, reduced loss of valuable reactants, H₂ and carbon oxides, and reduced cost and energy consumption of the means to separate H₂ from the purge.

### DETAILED DESCRIPTION OF THE INVENTION

In the following the invention will be further described with reference to the accompanying drawing. The drawing is exemplary and is not to be construed as limiting for the invention.

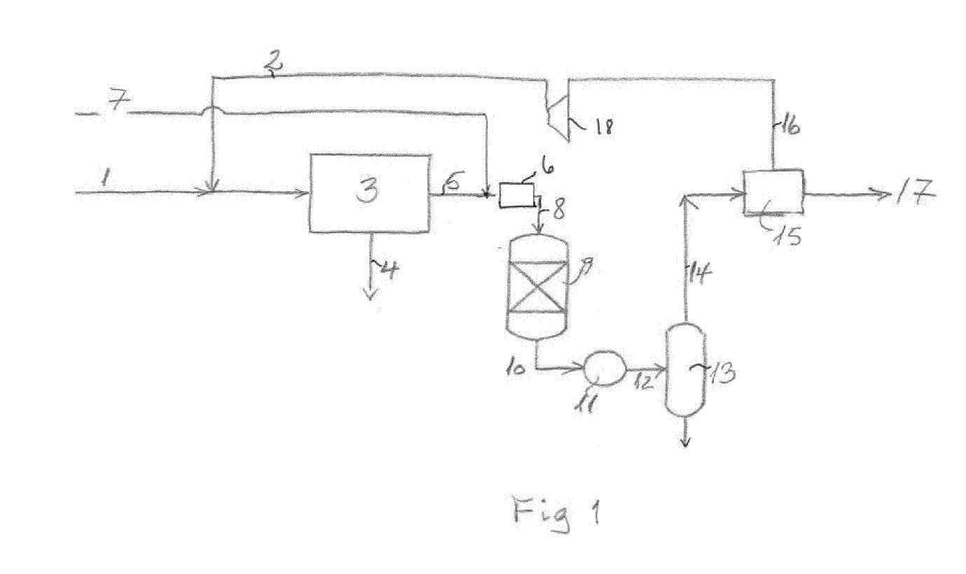

FIG. 1 shows a plant according to the present invention. Synthesis gas 1 is mixed with H₂ rich recycle gas 2 and passed to the synthesis section 3. From the synthesis section 3 a product stream 4 and a purge stream 5 are withdrawn. The purge stream is heated in preheater 6 and mixed with process steam 7 to obtain a mixed stream 8. Stream 8 is passed to a shift conversion unit 9, where steam and CO react to H₂ and CO₂. The reacted gas 10 is cooled to below its dew point in cooler 11. The cooled stream 12 is passed to a process condensate separator 13. The vapor stream 14 from the condensate separator 13 is passed to a gas separation unit 15. From the gas separation unit a H₂-enriched stream 16 and a H₂-depleted waste gas stream 17 are withdrawn. The H₂-enriched gas 16 may be compressed in the recycle compressor 18 to form the H₂ enriched recycle stream 2, which is added to the synthesis gas 1 as described above. If the synthesis gas comes from a synthesis gas compressor, the H₂- enriched gas 16 may also be added to the synthesis gas before this compressor. Alternatively or in combination, the synthesis gas and the H₂-enriched gas may be added separately to the synthesis section at different points in the process.

### Example

In a plant designed and operating according to the invention as described above and exemplified the accompanying FIG. 1, a total production of 1000 metric tons (MTPD) of methanol is produced when the plant is fed with 89090 Nm3/h of a synthesis gas containing 63.66 vol % H2, 1.63 vol % H2, 27,40 vol % CO, 4,42 vol % CO2, 0,36 vol % Ar, and 2,43 vol % CH4.

When a similar unit designed without the steps of treating the purge gas is fed with the same synthesis gas as the unit designed and operating according to the invention, a total production of 987.4 MTPD of methanol is obtained. I.e. the present process of treating the purge gas including heating, adding steam, converting CO and steam to CO₂ and H₂, cooling and separating condensate, all before admitting the gas to the gas separation unit, leads to an increased methanol production.

1. A process comprising the steps of
providing a purge stream from a synthesis section,
preheating at least part of said purge stream,
adding steam to the preheated purge stream to obtain a first mixed stream,
passing the first mixed stream through a shift conversion step thereby obtaining a conversion product stream, p1 passing at least part of the conversion product stream through a H₂ separation step producing a H₂ enriched stream and a H₂ depleted waste stream, and
returning at least part of said H₂ enriched stream to or upstream the synthesis section.

2. Process according to claim 1 comprising the step of cooling at least part of the conversion product stream before passing the cooled conversion product stream through a process gas separator thereby obtaining a condensate stream and a vapor stream, and passing at least part of the vapor stream to the H₂ separation step.

3. Process according to claim 1, wherein the H₂ depleted stream is used as fuel in fired heaters in the process unit and/or for power production.

4. Process according to claim 1, wherein the H₂ separation step is carried out in a membrane unit or in a PSA.

5. Plant comprising a synthesis section and a purge gas treatment line, said purge gas treatment line comprising:
preheater,
steam addition means,
shift conversion unit, and
H₂ separation means.

6. Plant according to claim 5 comprising a cooler and a process condensate separator upstream the H₂ separation means.

7. Plant according to claim 5, wherein the plant is arranged to carry out a process comprising the steps of:
providing a purge stream from a synthesis section,
preheating at least part of said purge stream,
adding steam to the preheated purge stream to obtain a first mixed stream,
passing the first mixed stream through a shift conversion step thereby obtaining a conversion product stream,
passing at least part of the conversion product stream through a H₂ separation step producing a H₂ enriched stream and a H₂ depleted waste stream, and
returning at least part of said H₂ enriched stream to or upstream the synthesis section.
